(19) United States
(12) Patent Application Publication
(10) Pub. No.: US 2025/0139955 A1
(43) Pub. Date: May 1, 2025

(54) METHOD AND A SYSTEM FOR IDENTIFICATION OF CERVICAL CANCER CELLS

(71) Applicant: ALCID Sp. z o.o, Warszawa (PL)

(72) Inventor: Artur OLSZEWSKI, Warszawa (PL)

(21) Appl. No.: 18/384,860

(22) Filed: Oct. 29, 2023

(51) Int. Cl. G06V 10/778; G06T 7/00; G06V 10/94; G06V 20/69; G16H 15/00

(52) U.S. Cl. CPC G06V 10/7788; G06T 7/0012; G06V 10/945; G06V 20/695; G06V 20/698; G16H 15/00; G06T 2200/24; G06T 2207/10056; G06T 2207/20021; G06T 2207/20076; G06T 2207/20081; G06T 2207/20084; G06T 2207/20104; G06T 2207/30024; G06T 2207/30168; G06T 2207/30242; G06V 2201/03

(57) ABSTRACT

A method for identifying cervical cancer cells, including: receiving a microscopic image of a specimen imaging a plurality of cells; analyzing a quality of the microscopic image; classifying the cells imaged on the microscopic image to indicate potentially cancerous cells; analyzing the whole microscopic image to determine an overall probability that the microscopic image comes from a potentially cancerogenous patient; presenting, on a single screen of a graphical user interface of a computer system: an overall image representing box configured to display the microscopic image with a zoom in and zoom out functionality; and a plurality of cell identification boxes configured to display enlarged images of the potentially cancerogenous cells; receiving via the graphical user interface an expert input; and generating a final report that identifies an overall probability that the microscopic image comes from a potentially cancerogenous patient.

### TECHNICAL FIELD

The present invention relates to computer-implemented identification of cervical cancer cells.

### BACKGROUND

Millions of women are tested for cervical cancer every year. This test is typically performed by visualizing cell samples from the cervix under a microscope to search for abnormal/atypical cells. A typical sample has from about 10,000 to about 50,000 cells that need to be quickly evaluated by a medical professional to identify these abnormal cells and perform the diagnosis. A single cell can be enough to classify the sample as cancerogenous. With so many cells, fast and accurate diagnosis is not possible, with medical professionals typically spending ˜30-60 minutes for each diagnosis.

### SUMMARY OF THE INVENTION

There is a need to provide a computer-implemented system that would facilitate identification of cervical cells, by identifying cells with features that indicate a high probability of the cell being abnormal.

In one aspect, the present invention relates to a computer-implemented method for identifying cervical cancer cells. The method comprises receiving a microscopic image of a specimen imaging a plurality of cells; analyzing a quality of the microscopic image; detecting that the quality of the microscopic image is satisfactory; in response to detecting that the quality of the image is satisfactory, classifying the cells imaged on the microscopic image to indicate potentially cancerous cells, wherein each potentially cancerous cell is assigned with a type descriptor indicating a cell type and a type probability indicating a probability that the cell is of a particular type. The method further comprises analyzing the whole microscopic image to determine an overall probability that the microscopic image comes from a potentially cancerogenous patient, based on distribution of the potentially cancerogenous cells. The method also comprises presenting, on a single screen of a graphical user interface of a computer system: an overall image representing box configured to display the microscopic image with a zoom in and zoom out functionality; and a plurality of cell identification boxes configured to display enlarged images of the potentially cancerogenous cells. Further, the method comprises receiving on said single screen of the graphical user interface an expert input, wherein the expert input indicates correction of cell type descriptors and type probabilities for at least some of the potentially cancerogenous cells; repeating the step of analyzing the whole microscopic image to determine an overall probability that the microscopic image comes from a potentially cancerogenous patient, based on distribution of the potentially cancerogenous cells, including the cell descriptors and probabilities corrected via the expert input; and generating a final report that identifies an overall probability that the microscopic image comes from a potentially cancerogenous patient.

The method may further comprise, after analyzing a quality of the microscopic image, detecting that the quality of the microscopic image is not satisfactory and in response to detecting that the quality of the image is not satisfactory, outputting an indication that the image is not diagnostic.

The step of analyzing the quality of the microscopic image may comprise: counting cells within the whole microscopic image to determine a number of cells imaged within the microscopic image; dividing the microscopic image into fragments and performing at least one of the following tests per at least some fragments: determining whether the particular fragment is in focus; and determining whether the particular fragment contains an object that is non-diagnostic; classifying the microscopic image as satisfactory if: the number of cells imaged within the microscopic image is higher than a cells number threshold; an area of image containing cells that is in focus is higher than a focus threshold; and an area of the image containing non-diagnostic objects is lower than a non-diagnostic area threshold.

The graphical user interface may further comprise a cell counter box configured to indicate the number of potentially cancerogenous cells corresponding to a particular type descriptor.

The graphical user interface may further comprise a summary diagnosis box to indicate the overall probability that the microscopic image comes from a potentially cancerogenous patient.

The method may further comprise performing the step of classifying the cells by means of a cells classifier that is an artificial intelligence module and training the cells classifier based on received expert input.

The final report may further identify a list of potentially cancerogenous cells along with the type descriptor and the type probability.

In another aspect, the invention is related to a computer-implemented system comprising at least one non-transitory processor-readable storage medium that stores at least one of processor-executable instructions or data and at least one processor communicably coupled to at least one non-transitory processor-readable storage medium and configured to perform the steps of the method as described herein.

These and other features, aspects and advantages of the invention will become better understood with reference to the following drawings, descriptions and claims.

### BRIEF DESCRIPTION OF DRAWINGS

Various embodiments are herein described, by way of example only, with reference to the accompanying drawings, wherein:

FIG. 1 shows an example microscopic image to be analyzed;

FIG. 2 shows an overview of a method for identification of cervical cancer cells;

FIG. 3 shows an overall structure of a computer-implemented system for identification of cervical cancer cells;

FIG. 4A shows a procedure for analyzing the quality of the microscopic image;

FIG. 4B shows fragmentation of the microscopic image and indication of inappropriate fragments;

FIGS. 5A, 5B show plots of probability density functions;

FIG. 6 shows a graphical user interface for presenting results to the user;

FIG. 7 shows an overall structure of a computer system to perform the method presented herein.

### DETAILED DESCRIPTION

The following detailed description is of the best currently contemplated modes of carrying out the invention. The description is not to be taken in a limiting sense but is made merely for the purpose of illustrating the general principles of the invention.

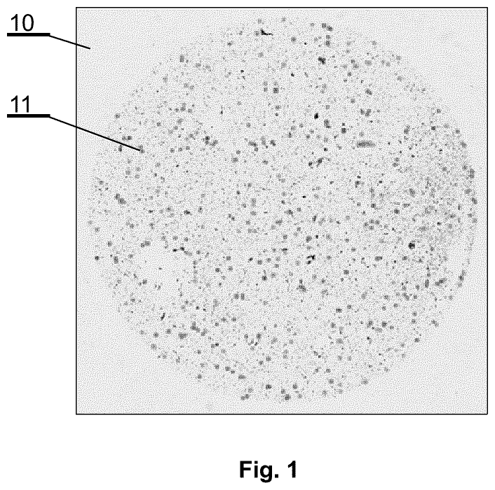

FIG. 1 shows an example of a microscopic image 10 to be analyzed. This kind of image can be obtained by scanning samples that are analyzed by microscoping. The step of collecting a specimen and imaging is performed by using known methods. The microscopic image 10 comprises a plurality (typically, tens of thousands) of cells 11, some of which may be cancerogenous and shall be identified.

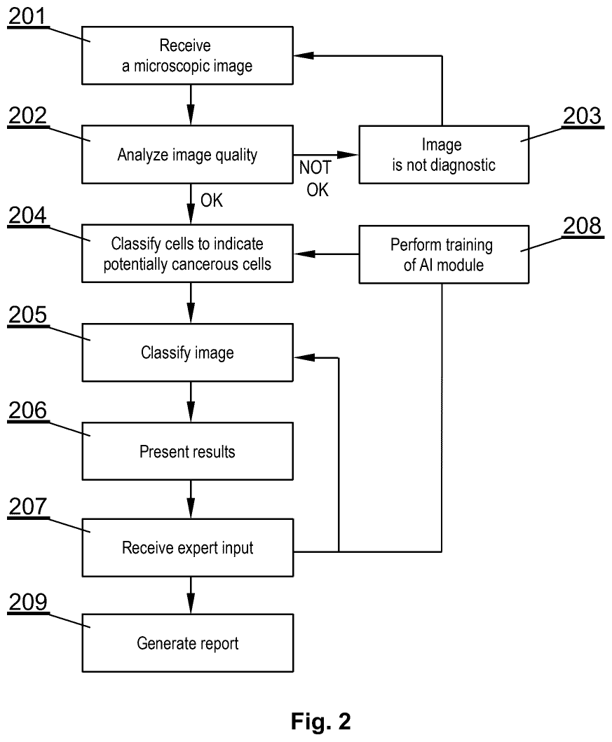

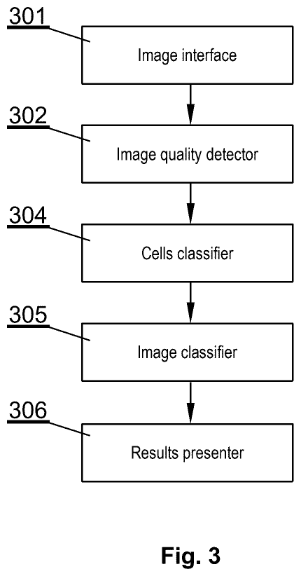

The method of the invention comprises the steps shown in an overview on FIG. 2, performed by means of modules shown in FIG. 3. First, in step 201, a microscopic image is read via an image interface 301, for example from a server to which data from a testing laboratory is registered. Preferably, high resolution images are used, e.g., having a resolution of 100,000×100,000 pixels.

Next, in step 202 the microscopic image is analyzed for quality by an image quality detector 302, as explained in detail in FIG. 3. If the quality is not satisfactory, then an indication that the image is not diagnostic is output in step 203, for example sent back to the source from which the microscopic image was received.

In step 204 the microscopic image is input to a cells classifier module 304 that is configured to detect cells and cell clusters to indicate within the image at least the cells that are considered as potentially cancerous, as will be explained below.

In step 205 the whole image is analyzed by an image classifier module 305.

Finally, in step 206 the results of the analysis are presented by means of a results presenter module 306.

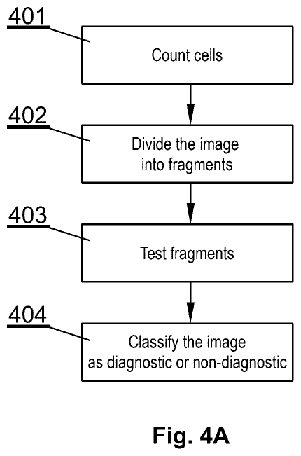

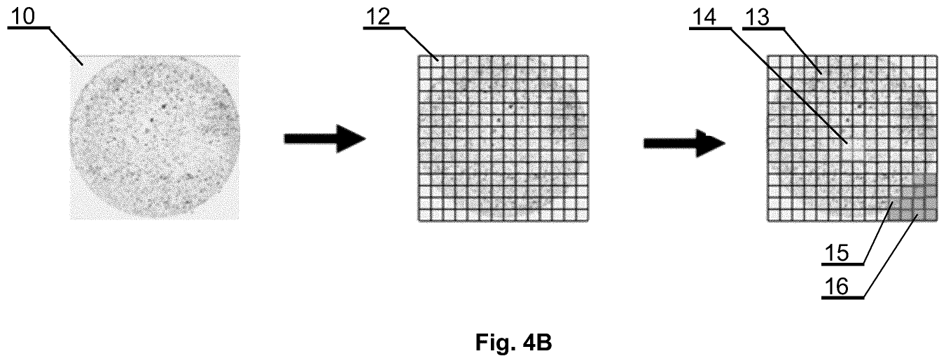

The image quality detector 302 used in step 202 is configured to initially check whether the image is of a sufficient quality to perform cell classification in subsequent steps, according to the procedure of FIG. 4A.

Initially, in step 401 the whole image is analyzed to perform cell counting, to determine how many cells are imaged within the microscopic image; this can be done, for example, by using YoloR algorithm to label all cells on the image.

Next, the microscopic image 10 is divided in step 402, as shown in FIG. 4B, into a grid of fragments 12 (for example, a grid of 1024×1024 fragments).

Next, in step 403 each fragment 12 is initially tested according to at least one of the following tests:
blur detection: to determine whether the particular fragment is in focus; this can be done, for example, by determining the features of a selected part of the image measuring the degree of its blurring, e.g. Laplacian variance, the modulus of the gradient value of the image, components from Sobel filter, etc. (based on these data, a classifier is built)—the fragment is classified as in focus if the blur is not higher than an allowable blur threshold;
foreign objects detection: to determine whether the fragment contains an object that does not seem to be a cell; this can be done, for example, by a neural network trained to detect non-diagnostic objects.
   As an outcome, the fragments can be indicated as:
Fragments 14 containing correctly imagined cells;
Fragments 15 containing foreign objects;
Fragments 16 which are blurred;
Fragments 17 with no cells.
   The microscopic image is classified in step 404 as diagnostic (i.e., appropriate for further processing to obtain conclusive results) if it meets the following criteria:
number of counted cells higher than a cells number threshold (e.g., higher than 10,000);
a predetermined percentage of image area containing cells being in focus, i.e. an area of image containing cells that is in focus is higher than a focus threshold (e.g., over 90%, or over 95%, or over 99% depending on system configuration);
foreign objects taking no more than a predefined area of the image, i.e. an area of the image containing non-diagnostic objects is lower than a non-diagnostic area threshold (e.g., less than 10%).

Step 202 can be performed relatively quickly and substantially in real time as soon as the microscopic image is received. Consequently, the system can provide a real-time response to received image and in case the image is not classified as diagnostic, a response can be sent back in step 203, which may prompt the facility that takes the image to take another image of the specimen again. Thereby, substantial time can be saved in the whole process, as the person preparing the image of the specimen can receive prompt response that the image should be corrected. The sample can be prepared again or even another sample can be taken to ensure that the patient gets a conclusive diagnosis after the visit to collect the sample.

The cells classifier 304 used in step 204 can be implemented as an artificial intelligence module. For example, this can be a convolutional neural network trained with expert-annotated images of cells and their corresponding descriptors. Alternatively, this can be a transformers-based network or any other computer vision-based algorithms. Once trained, the neural network can perform an inference process to receive an image comprising one or more cells and indicate a type descriptor (or more than one type descriptor) to which that cell is best matched. Along with a descriptor, a probability score can be provided that indicates the probability corresponding to that cell being of the determined type.

The cells classifier 304 can be configured to analyze the whole image or image fragments, for example fragments as used by the image quality detector or smaller fragments, even such small as to contain only a single cell. The methods for dividing an image into smaller fragments to facilitate recognition of small objects on the image are well known in the art of image recognition and do not need further clarification.

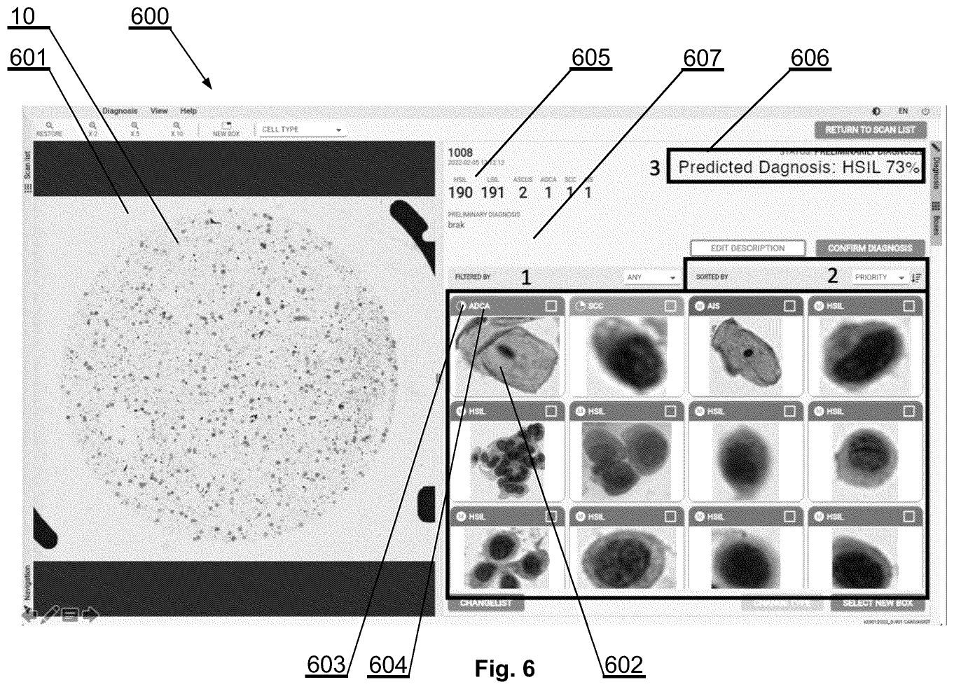

The cells can be classified according to templates ad glandular and squamous with level of dysplasia or reactive atypia. Based on this, the results can be further analyzed by an expert, for example via the user interface such as shown in FIG. 6, and final diagnosis can be made. This expert module indicates what is type of the cells and level of dysplasia is necessary for each class of Bethesda testing as following:
Atypical squamous cells
Atypical squamous cells of undetermined significance (ASC-US)
Atypical squamous cells—cannot exclude HSIL (ASC-H)
Low-grade squamous intraepithelial lesion (LGSIL or LSIL)
High grade squamous intraepithelial lesion (HGSIL or HSIL)
Squamous cell carcinoma
Atypical Glandular Cells not otherwise specified (AGC-NOS)
Atypical Glandular Cells, suspicious for AIS or cancer (AGC-neoplastic)
Adenocarcinoma in situ (AIS)

Each cell that is defined as potentially cancerous is assigned a type descriptor indicating the type of cell and a type probability that the cell is of that type (wherein that information along with an image of the cell is presented in areas 602, 603, 604 of FIG. 6).

The image classifier 305 used in step 205 is configured to perform an overall diagnosis of the microscopic image, based on results of step 204 wherein individual cells were diagnosed and some of them indicated as abnormal. Typically, a few percent of cells are diagnosed as abnormal, however most of them are diagnosed with a low probability.

The aim of step 205 is to determine what is an overall probability that the microscopic image sample comes from a potentially cancerogenous patient.

One approach of implementing the image classifier 305 can be to use the features from the distribution of the potentially cancerogenous cells. In particular, the classification is performed by analyzing the probability density function of the distribution of the cell abnormality scores for the whole image. For example, if a slide has about 10,000 cells (the number of cells is detected in step 204), then typically from 10 to 500 of the cells will be identified as abnormal with a probability score in the range between >0.0 and <=1.0. The remaining cells that have not been classified as abnormal are assigned a probability score of 0.0. The probability density function determines the number of cells having a specific probability of being abnormal, i.e., it specifies the density (i.e., number of cells with this score over the total number of cells identified) of each slice denoting the value of each feature. The function is then input into a classifier such as an xgboost binary classifier for each of the healthy or cancerogenous slides, wherein the classifier is configured to output a probability of that a sample is healthy or cancerogenous.

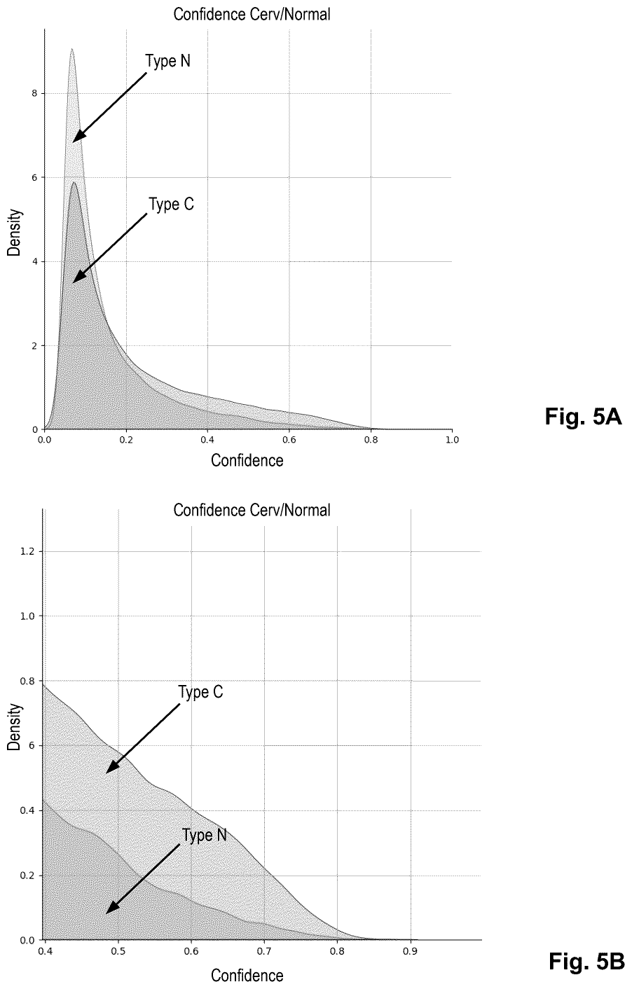

FIGS. 5A-5B indicate examples of probability density functions for: type C-cancer, N-normal, wherein at FIG. 5A the confidence range is from 0.1-1.0, and at FIG. 5B the confidence range is from 0.4-1.0.

Alternatives to the above presented classifier might include deep neural networks that consider the aggregate features of the images with highest probabilities or aggregate features of all detected cell images.

Finally, the image presenter 306 is used in step 206 to present results of the earlier performed steps.
FIG. 6 presents a graphical user interface 600, wherein the following information is presented on screen.

An overall image representing box 601 is provided to display the microscopic image, wherein the user is able to zoom in and out of the image and move the image within the representation window, in order to be able to manually verify details of a desired image fragment.

A plurality of cell identification boxes 602 are provided to display enlarged images of cells for which there is a high probability that the cell is of an abnormal type. Each box 602 may comprise a heading with an icon 603 representing the type probability assigned to that cell (e.g. in a form of a pie chart or a letter M to indicate that the cell has to be classified manually) and a header 604 indicating the predicted type descriptor of that cell (in addition, the color of the header tab may be matched to a particular cell type so that particular cell types are easier to spot for the user). The cell identification boxes 602 can be presented in order from the most dysplastic, suspicious cells to the most normal cells.

Further, a cell counter box 605 is provided to indicate the number of cells that have been detected on that image as abnormal (i.e., potentially cancerogenous cells). For example, the following cell types can be indicated:
HSIL—High grade squamous intraepithelial lesion
LSIL—Low-grade squamous intraepithelial lesion
ASCUS—Atypical squamous cells of undetermined significance
ADCA—Adenocarcinoma
SCC—Squamous cell carcinoma
AIS—Adenocarcinoma in situ

Moreover, a summary diagnosis box 606 is provided to indicate the predicted diagnosis for the whole microscopic image, i.e., the overall probability that the microscopic image (10) comes from a potentially cancerogenous patient as predicted in step 205.

Next, expert input is received in step 207, namely the user (a skilled medical professional) may review the cell identification boxes 602 to confirm whether the cells imaged therein are actually the cells of the predicted type and confirm the diagnosis to increase the probability score for that cell (e.g. to 100% or to a lower value if the user is not 100% sure, or to 0% if the cell is a normal cell). Moreover, the user may assign manually cell types and probabilities to cell images which have been indicated as potentially abnormal, but without any type assigned. Upon selection of a particular cell identification box 602, the overall microscopic image in box 601 may be adapted to mark the point at which that cell is located or even enlarged to show the enlarged image of that cell and surrounding cells.

A summary box 607 allows the user to manually enter description and diagnosis for the analyzed microscopic image.

If the user corrected cell type descriptors or type probabilities, the image classifier 305 is run again to determine a new diagnosis based on the verified cell types. If the user changed the cell type for a particular cell image, that cell image may be used in step 208 to train the cells classifier module 304 to increase its inference capabilities.

The design of the user interface is modelled on the principle of machine teaching. The design is configured such as to ensure that it is easy for the user to see and modify the computer-generated predictions. It aims to entice the user to modify the predictions to see the updated probability score for the entire slide. Moreover, the predictions are sorted by default based on probability of being anomalous. This allows the user to see quickly the most suspicious cells but also provides the machine learning classifier with the most information if the user changes the classification of these cells. This is because the highest probability cells that have been misassigned would contribute the most to the loss function.

Combining this information from user-generated labels that are easy for them to generate with retraining allows to significantly improve the model quality.

Therefore, the presented graphical user interface allows the user to efficiently sort and visualize the most essential information on the cells predicted as being abnormal within the microscopic image. This increases the efficiency of image analysis by the user, while still allowing the user to manually review the image. This combination of the visualization of the results from the cell and image classifiers with the secondary evaluation based on human input of only a small subset of cells allows to speed up the evaluation and provide the medical professional required confidence in the final diagnosis.

Finally, when the expert user does not provide any more manual input on correction of data, in step 209 a final report is generated that identifies an overall probability that the microscopic image 10 comes from a potentially cancerogenous patient and optionally a list of potentially cancerogenous cells along with the type descriptor and the type probability and optionally the number of potentially cancerogenous cells corresponding to a particular type descriptor. The final report may have a form such as shown in FIG. 6 or another form containing said information.

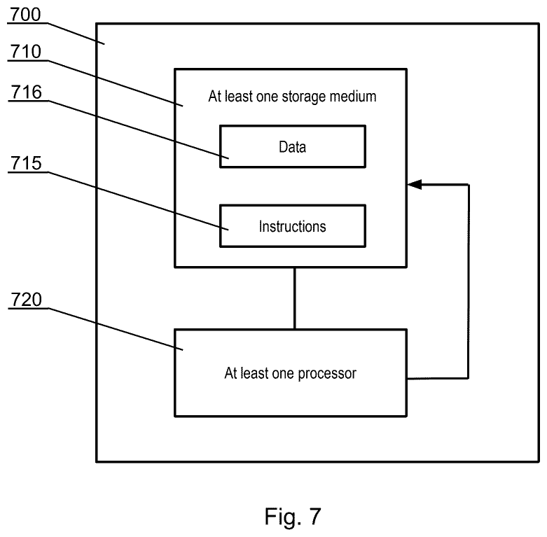

The functionality described herein can be implemented in a computer-implemented system 700, such as shown in FIG. 7. The system may include at least one non-transitory processor-readable storage medium 710 that stores at least one of processor-executable instructions 715 or data 716 and at least one processor 720 communicably coupled to at least one non-transitory processor-readable storage medium. At least one processor is configured to perform the steps of the method of FIG. 2 to implement the functionality of the modules presented in FIG. 3.

While the invention has been described with respect to a limited number of embodiments, it will be appreciated that many variations, modifications and other applications of the invention may be made. Therefore, the claimed invention as recited in the claims that follow is not limited to the embodiments described herein.

1. A computer-implemented method for identifying cervical cancer cells, the method comprising:
receiving a microscopic image of a specimen imaging a plurality of cells;
analyzing a quality of the microscopic image;
detecting that the quality of the microscopic image is satisfactory;
in response to detecting that the quality of the image is satisfactory, classifying the cells imaged on the microscopic image to indicate potentially cancerous cells, wherein each potentially cancerous cell is assigned with a type descriptor indicating a cell type and a type probability indicating a probability that the cell is of a particular type;
analyzing the whole microscopic image to determine an overall probability that the microscopic image comes from a potentially cancerogenous patient, based on distribution of the potentially cancerogenous cells;
presenting, on a single screen of a graphical user interface of a computer system:
an overall image representing box configured to display the microscopic image with a zoom in and zoom out functionality; and
a plurality of cell identification boxes configured to display enlarged images of the potentially cancerogenous cells;
receiving on said single screen of the graphical user interface an expert input, wherein the expert input indicates correction of cell type descriptors and type probabilities for at least some of the potentially cancerogenous cells;
repeating the step of analyzing the whole microscopic image to determine an overall probability that the microscopic image comes from a potentially cancerogenous patient, based on distribution of the potentially cancerogenous cells, including the cell descriptors and probabilities corrected via the expert input; and
generating a final report that identifies an overall probability that the microscopic image comes from a potentially cancerogenous patient.

2. The method according to claim 1, further comprising, after analyzing a quality of the microscopic image, detecting that the quality of the microscopic image is not satisfactory and in response to detecting that the quality of the image is not satisfactory, outputting an indication that the image is not diagnostic.

3. The method according to claim 1, wherein the step of analyzing the quality of the microscopic image comprises:
counting cells within the whole microscopic image to determine a number of cells imaged within the microscopic image;
dividing the microscopic image into fragments and performing at least one of the following tests per at least some fragments:
determining whether the particular fragment is in focus; and
determining whether the particular fragment contains an object that is non-diagnostic;
classifying the microscopic image as satisfactory if:
the number of cells imaged within the microscopic image is higher than a cells number threshold;
an area of image containing cells that is in focus is higher than a focus threshold; and
an area of the image containing non-diagnostic objects is lower than a non-diagnostic area threshold.

4. The method according to claim 1, wherein the graphical user interface further comprises a cell counter box configured to indicate the number of potentially cancerogenous cells corresponding to a particular type descriptor.

5. The method according to claim 1, wherein the graphical user interface further comprises a summary diagnosis box to indicate the overall probability that the microscopic image comes from a potentially cancerogenous patient.

6. The method according to claim 1, comprising performing the step of classifying the cells by means of a cells classifier that is an artificial intelligence module and training the cells classifier based on received expert input.

7. The method according to claim 1, wherein the final report further identifies a list of potentially cancerogenous cells along with the type descriptor and the type probability.

8. A computer-implemented system comprising at least one non-transitory processor-readable storage medium that stores at least one of processor-executable instructions or data and at least one processor communicably coupled to at least one non-transitory processor-readable storage medium and configured to perform the steps of the method according to claim 1.
